(19) United States
(12) Patent Application Publication
(10) Pub. No.: US 2021/0264129 A1
(43) Pub. Date: Aug. 26, 2021

(54) PUPIL FEATURE EXTRACTION APPARATUS, PUPIL FEATURE EXTRACTION METHOD, AND PROGRAM

(71) Applicant: NIPPON TELEGRAPH AND TELEPHONE CORPORATION, Tokyo (JP)

(72) Inventors: Shimpei YAMAGISHI, Tokyo (JP); Makoto YONEYA, Tokyo (JP); Shigeto FURUKAWA, Tokyo (JP)

(73) Assignee: NIPPON TELEGRAPH AND TELEPHONE CORPORATION, Tokyo (JP)

(21) Appl. No.: 17/267,421

(22) PCT Filed: Jul. 10, 2019
(86) PCT No.: PCT/JP2019/027248

§ 371 Date: Feb. 9, 2021

(30) Foreign Application Priority Data
Aug. 13, 2018 (JP) 2018-152192

(51) Int. Cl. G06K 9/00; G06T 7/60

(52) U.S. Cl. CPC G06K 9/0061; G06T 2207/30201; G06T 7/60

(57) ABSTRACT

Provided is a technology to extract a feature value related to the pupil size that is not susceptible to the positional relationship between a camera and an eyeball. The technology includes: a pupil information acquisition unit that acquires pupil information expressing a pupil size of a subject from an image of an eyeball of the subject; an iris information acquisition unit that acquires iris information expressing an iris size of the subject from the image of the eyeball of the subject; and a pupil feature value calculation unit that calculates a ratio of the pupil information to the iris information as a pupil feature value.

### TECHNICAL FIELD

The present invention relates to a technology to extract a feature value related to the pupil size.

### BACKGROUND ART

It has been known that the pupil size changes according to the brightness of a region at which a person is gazing or a psychological state. By the use of a change in pupil size, the degree of saliency of a sound can be, for example, estimated (Cited Patent Literature 1).
(Cited Patent Literature 1: Japanese Patent Application Laid-open No. 2015-132783).

For the estimation of a change in pupil size used in Patent Literature 1, a dedicated device (Non-Patent Literature 1) called an eyeball movement measurement device can be, for example, used.

### CITATION LIST

### Non Patent Literature

[NPL 1] tobii pro, [online], [Searched on Jun. 6, 2018] on the Internet <URL: https://www.tobiipro.com/ja/?gclid=EAIaIQobChMI9dzRgfq92wIVlYePCh2l1ge6EAAYASAAEgLqy_D_BwE>

### SUMMARY OF THE INVENTION

### Technical Problem

In a general eyeball movement measurement device, a pupil radius is measured using an image captured by a camera. According to the method, the shape of a pupil is captured in a distorted state depending on the positional relationship between a camera and an eyeball, and thus a pupil radius is measured as being apparently changed. For this reason, a pupil radius during saccade or a pupil radius in a case in which the position of a gaze is different cannot be, for example, correctly measured in some cases. That is, there is a problem that a change in pupil size cannot be correctly estimated in a case in which the positional relationship between a camera and an eyeball changes with time.

In view of this, the present invention has an object of providing a technology to extract a feature value related to the pupil size that is not susceptible to the positional relationship between a camera and an eyeball.

### Means for Solving the Problem

An aspect of the present invention includes: a pupil information acquisition unit that acquires pupil information expressing a pupil size of a subject from an image of an eyeball of the subject; an iris information acquisition unit that acquires iris information expressing an iris size of the subject from the image; and a pupil feature value calculation unit that calculates a ratio of the pupil information to the iris information as a pupil feature value.

### Effects of the Invention

According to the present invention, it is possible to extract a feature value related to the pupil size that is not susceptible to the positional relationship between a camera and an eyeball.

### BRIEF DESCRIPTION OF DRAWINGS

FIG. 1 is a diagram showing the state of an experiment.

FIGS. 2(A) and 2(B) are diagrams showing experimental results.

FIG. 3 is a diagram showing experimental results.

FIG. 4 is a block diagram showing an example of the configuration of a pupil feature value extraction apparatus 100.

FIG. 5 is a flowchart showing an example of the operation of the pupil feature value extraction apparatus 100.

FIGS. 6(A) and 6(B) are diagrams describing an edge extraction algorithm.

FIG. 7 is a diagram expressing a change in pupil size.

FIG. 8 is a block diagram showing an example of the configuration of a sound saliency estimate apparatus 200.

FIG. 9 is a flowchart showing an example of the operation of the sound saliency estimate apparatus 200.

FIG. 10 is a diagram describing a time Tₐ at which a speed becomes maximum and a rising time Tₚ.

### DESCRIPTION OF EMBODIMENTS

Hereinafter, embodiments of the present invention will be described in detail. Note that constituting units having the same functions will be denoted by the same numbers and their duplicated descriptions will be omitted.

### Technological Background

The pupil size changes due to various factors. For example, a pupil changes due to the brightness of a visual input (light reflex) or an internal factor such as the degree of concentration on a task or an emotional state. Further, as described above, the pupil size apparently changes due to a geometric factor such as the positional relationship between a camera and an eyeball.

On the other hand, it is presumed that the iris size does not change due to the brightness of a visual input or an internal factor but apparently changes due to a geometric factor such as the positional relationship between a camera and an eyeball like a pupil.

In view of this, it is presumed that the use of the ratio of the pupil size to the iris size as a feature value related to the pupil size makes it possible to correctly estimate, even when the positional relationship between a camera and an eyeball changes with time, an actual change in size (a change due to light reflex or an internal factor) of the pupil while eliminating the influence of an apparent change due to the positional relationship between the camera and the eyeball.

Hereinafter, an experiment aimed at confirming the hypothesis that “the ratio of the pupil size to the iris size is not susceptible to a change in positional relationship between a camera and an eyeball” will be described.

[Experiment]

An image of a gaze point that serves as a visual sign is displayed on a display placed in front of a subject. Since the position of the gaze point moves left or right after a certain time, the subject is instructed to move his/her eyes so as to follow the position.

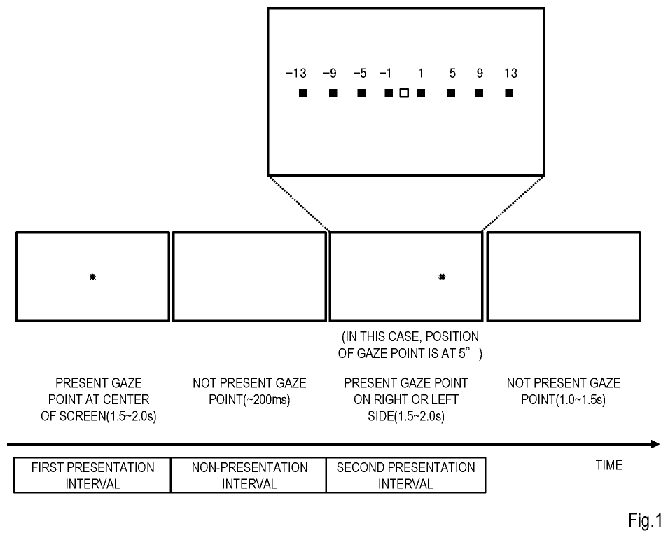

After the image of the gaze point is displayed at an initial position (central position) for a certain time and erased for a certain time, an image obtained by moving the position of the gaze point left or right is displayed (see FIG. 1). Here, a time interval in which the image of the gaze point is displayed at the initial position is called a “first presentation interval”, a time interval in which the image of the gaze point is erased is called a “non-presentation interval”, and a time interval in which the image of the gaze point after its movement is displayed is called a “second presentation interval”. The movements of the eyes of the subject in the first presentation interval and the second presentation interval are shot by a camera to measure a change in size of the eyes. Two cameras are used for shooting. A camera for measuring a right eye is put on the right side of the display, and a camera for measuring a left eye is put on the left side of the display.

Note that the image of the gaze point is caused to move from side to side within the range of −13° to 13°. The positional relationships between the cameras and the eyes (pupils or irises) change as the eyes move in the direction of the gaze point. That is, the degree of influence of a geometric factor on a change in size can be observed by the comparison between the sizes of the pupils or the irises for each gaze point.

[Experimental Results]

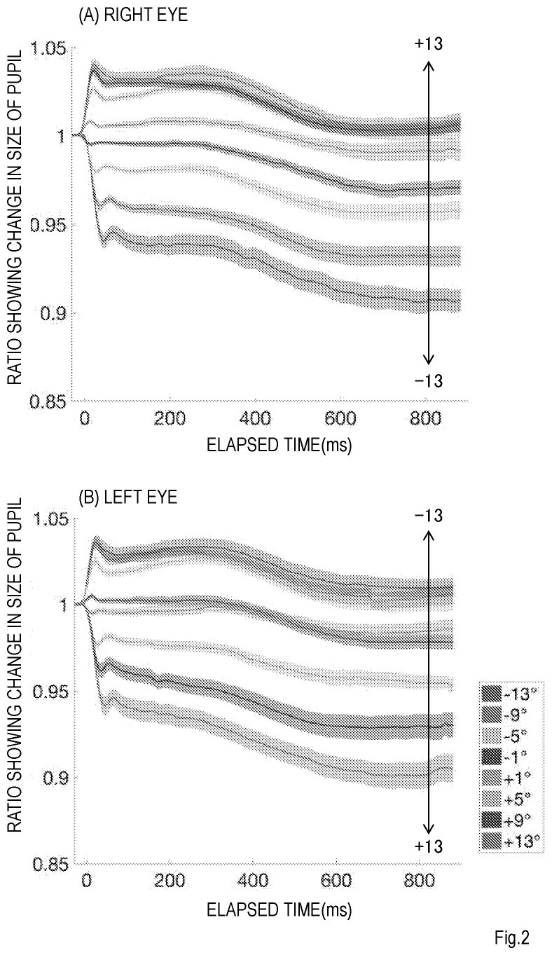

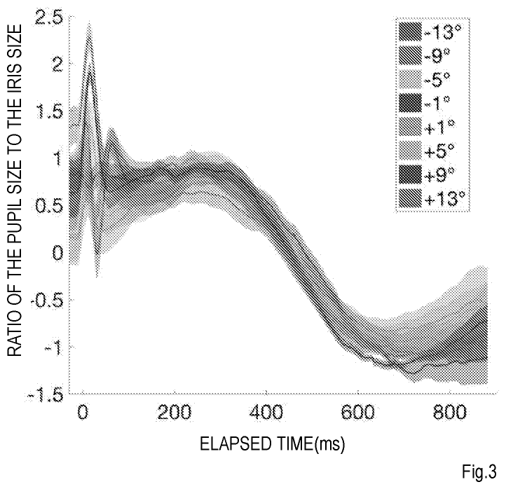

FIGS. 2(A) and 2(B) and FIG. 3 are diagrams showing experimental results. First, FIGS. 2(A) and 2(B) will be described. FIG. 2(A) shows a ratio showing a change in size of the pupil of the right eye, and FIG. 2(B) shows a ratio showing a change in size of the pupil of the left eye. These ratios express ratios relative to values just before the second presentation interval. It appears from the comparison between FIG. 2(A) and FIG. 2(B) that a ratio showing a change in size of the pupil of the right eye and a ratio showing a change in size of the pupil of the left eye have an opposite relationship with respect to a direction in which the image of the gaze point moves. That is, when the image of the gaze point moves in the right direction, the ratio showing the change in size of the pupil of the right eye increases while the ratio showing the change in size of the pupil of the left eye decreases. Further, when the image of the gaze point moves in the left direction, the ratio showing the change in size of the pupil of the right eye decreases while the ratio showing the change in size of the pupil of the left eye increases. This represents that the sizes of the pupils imaged by the cameras become apparently larger as the gaze directions and the directions of the cameras come closer to a parallel direction and become apparently smaller as the gaze directions are further separated from the front sides of the cameras (for example, an angle formed between the gaze direction and the direction of the camera comes closest to parallel when the image of the gaze point is presented at a position of 13° since the camera for measuring the right eye is put on the right side of the display). In this connection, the reason why the values of the ratio become 1 when an elapsed time is around 0 is that the subject sees the center of the display before moving his/her eyes.

Next, FIG. 3 will be described. FIG. 3 is a diagram showing a state in which the ratio of the pupil size to the iris size (the pupil size/the iris size) changes with time. As is clear from FIG. 3, there is no difference in the ratio (z-score) of the pupil size to the iris size depending on the position of a gaze. This is because an apparent change in the iris size and an apparent change in the pupil size seem to cancel each other when the ratio of the pupil size to the iris size is taken. Accordingly, the change in the pupil size can be evaluated by the use of the ratio regardless of the position of a gaze.

### First Embodiment

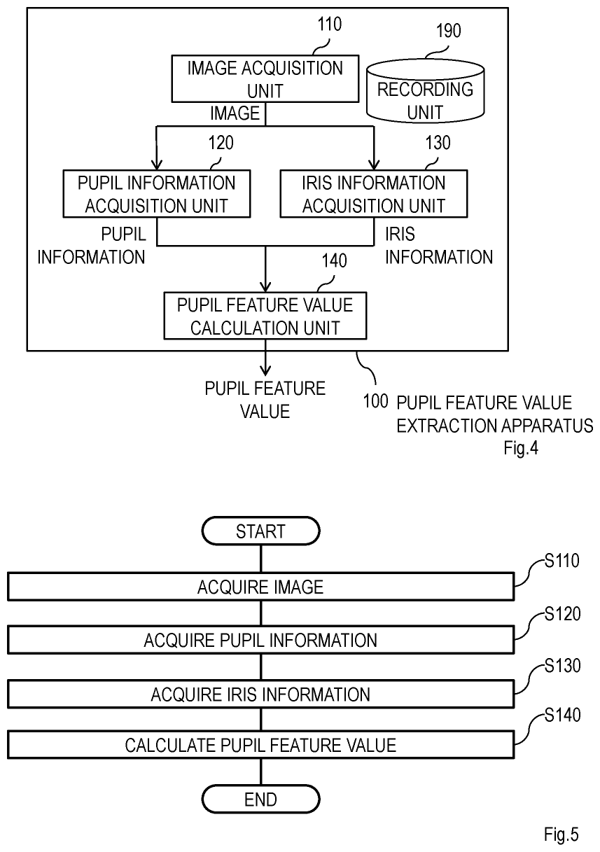

Hereinafter, a pupil feature value extraction apparatus 100 will be described with reference to FIGS. 4 and 5. FIG. 4 is a block diagram showing the configuration of the pupil feature value extraction apparatus 100. FIG. 5 is a flowchart showing the operation of the pupil feature value extraction apparatus 100. As shown in FIG. 4, the pupil feature value extraction apparatus 100 includes an image acquisition unit 110, a pupil information acquisition unit 120, an iris information acquisition unit 130, a pupil feature value calculation unit 140, and a recording unit 190. The recording unit 190 is a constituting unit that appropriately records information necessary for the processing of the pupil feature value extraction apparatus 100.

The operation of the pupil feature value extraction apparatus 100 will be described in accordance with FIG. 5.

[Image Acquisition Unit 110]

In S110, the image acquisition unit 110 acquires and outputs an image of an eyeball of a subject. As a camera used for image shooting, an infrared camera can be, for example, used. Note that the camera may be set to shoot both right and left eyeballs or only one of the eyeballs. In the following description, the camera is set to shoot only one of the eyeballs.

[Pupil Information Acquisition Unit 120]

In S120, the pupil information acquisition unit 120 acquires, using the image acquired in S110 as an input, pupil information expressing the pupil size of the subject from the image, and outputs the acquired pupil information. When a pupil radius (the radius of the pupil) is used as the pupil information, the radius of a circle fitted to a pupil region (a region corresponding to the pupil) in the image of the eyeball of the subject is only required to be used. Note that any value such as the area of the pupil and the diameter of the pupil besides the pupil radius can be used as the pupil information so long as the value expresses the pupil size.

[Iris Information Acquisition Unit 130]

In S130, the iris information acquisition unit 130 acquires, using the image acquired in S110 as an input, iris information expressing the iris size of the subject from the image, and outputs the acquired iris information. The acquisition of the iris information may be performed by the same method as that of the acquisition of the pupil information in S120 (however, it is difficult to perform circle fitting on the iris due to the influence of an eyelid compared with the case of the pupil. Therefore, it is presumed that another method is desirable in some cases (see a modification that will be described later)). Accordingly, any value such as an iris radius (the radius of the iris), the area of the iris, and the diameter of the iris can be used as the iris information so long as the value expresses the iris size.

[Pupil Feature Value Calculation Unit 140]

In S140, the pupil feature value calculation unit 140 calculates, using the pupil information acquired in S120 and the iris information acquired in S130 as inputs, the ratio of the pupil information to the iris information (the pupil information/the iris information) as a pupil feature value from the pupil information and the iris information, and outputs the calculated pupil feature value. Here, the pupil information and the iris information are preferably acquired by the same method. For example, when a pupil radius is used as the pupil information, an iris radius is used as the iris information.

Note that when images of both the right and left eyeballs are used, the processing of S120 to S140 is only required to be performed on the respective eyeballs.

According to the embodiment of the present invention, it is possible to extract a feature value that shows the pupil size, and that is not susceptible to the positional relationship between a camera and an eyeball.

<Modification>

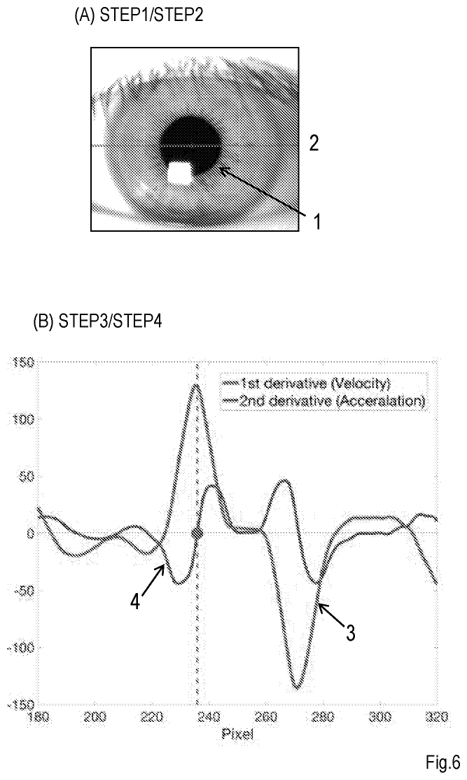

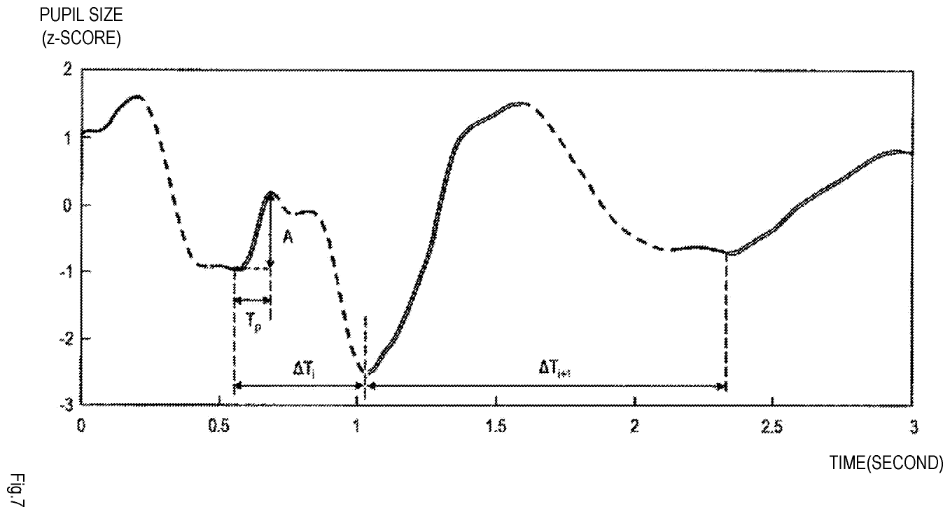

The pupil size or the iris size can be acquired by the use of points on the edge of a pupil region or an iris region in an image. Hereinafter, an algorithm (edge extraction algorithm) for extracting the edges of a pupil region or an iris region in an image will be described (see FIGS. 6(A) and 6(B)).

(Edge Extraction Algorithm)

Step 1: In a binary image obtained by converting a shot image of an eyeball of a subject, a region having intensity smaller than or equal to a prescribed threshold is extracted as a pupil region or an iris region. Note that the prescribed threshold is a value set for each subject and is a value different depending on whether the pupil region is extracted or the iris region is extracted.
Step 2: The gray value of pixels on a line (line in a horizontal direction in FIG. 6(A)) passing through the center of the pupil region or the iris region is calculated. Specifically, the average value of two vertical rows across the center of the pupil region or the iris region is calculated as the gray value of the pixels on the line passing through the center.
Step 3: The peak of the first derivative of the gray value is extracted. Note that when the peak is searched from left on the line passing through the above center, the peak of the first derivative becomes positive at a left edge and becomes negative at a right edge. This is because the peak is searched from a bright spot to a dark spot near the left edge and searched from a dark spot to a bright spot near the right edge. By the use of the information, the false detection of the peak can be reduced.
Step 4: The zero cross point (a circle in FIG. 6(B)) of the second derivative of the gray value near the peak extracted in step 3 is extracted as an edge. In the example of FIG. 6(B), a value of 235.8863 is extracted as the pixel value of the edge. By the use of the zero cross point of a second derivative as described above, it is possible to estimate an edge at a sub-pixel level.

The procedure of the steps 1 to 4 is performed so as to calculate two edges for each of the pupil region and the iris region. Accordingly, the above procedure is performed four times in total.

Finally, pupil information and iris information are only required to be calculated using the two edges for the pupil region and the two edges for the iris region. For example, the diameter of the pupil can be calculated by finding the difference between the values of the pixels of the two edges in the pupil region.

Accordingly, the pupil information acquisition unit 120 acquires, using the image acquired in S110 as an input, pupil information expressing the pupil size of a subject using two points on the edge of a pupil region in the image, and outputs the acquired pupil information. Similarly, the iris information acquisition unit 130 acquires, using the image acquired in S110 as an input, iris information expressing the iris size of the subject using two points on the edge of an iris region in the image, and outputs the acquired iris information.

### Second Embodiment

In the present embodiment, the degree of saliency of a sound is estimated on the basis of a change in pupil size. At this time, the change in the pupil size is extracted on the basis of a pupil feature value described in the first embodiment.

Note that the degree of saliency of a sound will also be called sound saliency in the following description. Further, a “sound having high saliency” includes not only a sound salient during careful listening but also a sound salient during unintentional listening.

First, a change in pupil size will be described. When a subject is gazing at a certain point, the pupil size does not remain constant but is changing. FIG. 7 is a diagram expressing a change in pupil size. In FIG. 7, a horizontal axis expresses a time (second), and a vertical axis expresses the size (z-score) of the pupil.

The pupil size expands (mydriasis) with a musculus dilator pupillae put under the control of a sympathetic nervous system and reduces (myosis) with a musculus sphincter pupillae put under the control of a parasympathetic nervous system. In FIG. 7, portions indicated by dashed lines express myosis, and portions indicated by double lines express mydriasis. A change in pupil size is mainly classified into the three responses of light reflex, convergency reflex, and a change due to emotion. The light reflex is a reaction in which the pupil size changes to control the amount of light input on the retina. Myosis occurs against strong light, while mydriasis occurs at a dark spot. The convergency reflex is a reaction in which a pupil radius changes as both eyes internally or externally roll (convergence movement) to adjust a focus. Myosis occurs when the eyes see a near side, while mydriasis occurs when the eyes see a far side. The change due to emotion is a reaction that occurs against external stress regardless of any of the above responses. Mydriasis occurs when a sympathetic nerve becomes dominant with anger, surprise, or active movements, while myosis occurs when a parasympathetic nerve becomes dominant under a relaxed condition.

For the perception of a salient sound as well, it is presumed that a sympathetic nerve becomes dominant with a feeling close to surprise and mydriasis occurs. Therefore, the features of mydriasis are more suitable for estimating the degree of saliency of a sound than those of myosis. In the present embodiment, a salient sound is estimated on the basis of the features of mydriasis among the changes of the pupil size.

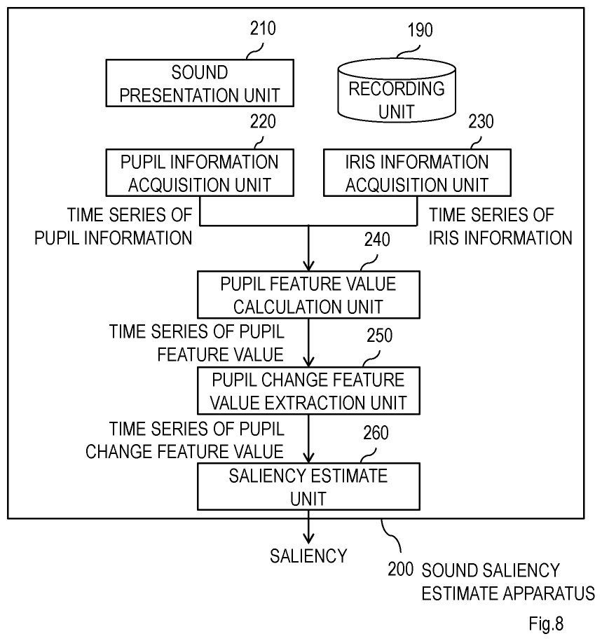

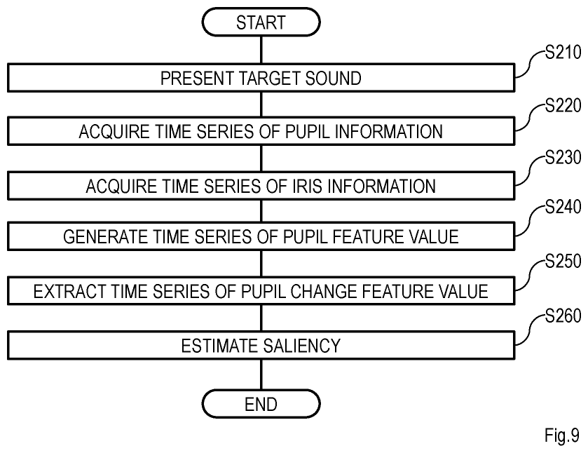

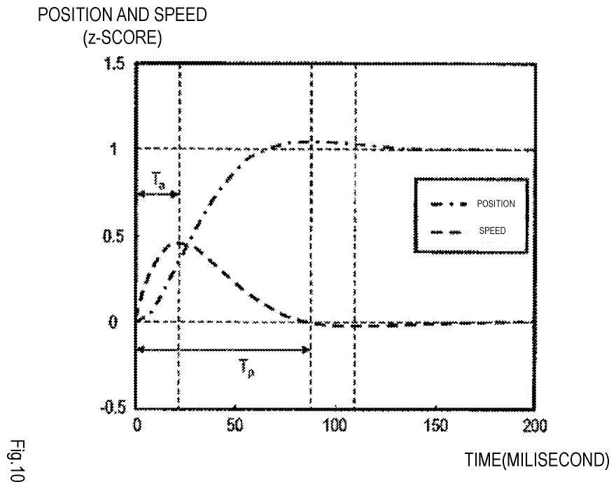

Hereinafter, a sound saliency estimate apparatus 200 will be described with reference to FIGS. 8 and 9. FIG. 8 is a block diagram showing the configuration of the sound saliency estimate apparatus 200. FIG. 9 is a flowchart showing the operation of the sound saliency estimate apparatus 200. As shown in FIG. 8, the sound saliency estimate apparatus 200 includes a sound presentation unit 210, a pupil information acquisition unit 220, an iris information acquisition unit 230, a pupil feature value calculation unit 240, a pupil change feature value extraction unit 250, a saliency estimate unit 260, and a recording unit 190. The recording unit 190 is a constituting unit that appropriately records information necessary for the processing of the sound saliency estimate apparatus 200.

The operation of the sound saliency estimate apparatus 200 will be described in accordance with FIG. 9.

[Sound Presentation Unit 210]

In S210, the sound presentation unit 210 presents a prescribed sound (a sound that is to be estimated and hereinafter also called a target sound) to a subject so as to be audible in a first time interval, and the above prescribed sound is not audible in a second time interval different from the first time interval. For example, the prescribed sound is presented at an audible volume by a headphone, a speaker, or the like in the first time interval. However, when the presentation time of the prescribed sound is short (about several tens of millimeter seconds or the like), up to several seconds in a time zone just after the presentation of the prescribed sound may be defined as the first time interval so as to include mydriasis in the first time interval so long as the condition that a sound other than the prescribed sound is not presented is satisfied. In the second time interval, a sound different from the prescribed sound may be presented to the subject so as to be audible, or no sound may be presented. Alternatively, even if a prescribed sound is output, a state in which the prescribed sound is not audible by the subject due to its extremely small volume is only required to be created. However, the second time interval is set so as not to overlap the first time interval and set as a time zone having the same length as the first time interval.

[Pupil Information Acquisition Unit 220]

In S220, the pupil information acquisition unit 220 acquires and outputs the time series of pupil information (hereinafter called the time series of first pupil information and the time series of second pupil information) that correspond to the first time interval and the second time interval, respectively, and express the pupil size of the subject. For example, when a pupil radius (the radius of the pupil) is used as the pupil size, the pupil radius is measured by an image processing method using an infrared camera. In the first time interval and the second time interval, the subject is caused to gaze at a certain point, and an image of the pupil at that time is captured using the infrared camera. Then, captured results are subjected to image processing to acquire the time series of the pupil radius for each time (at, for example, 1000 Hz). Note that the sizes of both right and left pupils may be acquired or the size of only any one of the pupils may be acquired. In the present embodiment, only the size of one of the pupils is acquired. For example, the radius of a circle fitted to the pupil is used with respect to the shot image. Further, since the pupil radius finely fluctuates, a value subjected to smoothing (smoothened) for each prescribed time interval may also be used. Here, the pupil size in FIG. 7 is expressed using a z-score obtained when the average of all the data of the pupil radius acquired for each time is 0 and the standard deviation thereof is 1, and the pupil radius is subjected to smoothing at an interval of about 150 ms. However, the pupil radius acquired by the pupil information acquisition unit 220 may not be the z-score but the value itself of the pupil radius or any value such as the area and the diameter of the pupil may be used so long as the value corresponds to the pupil size. In a case in which the area or the diameter of the pupil is used, an interval in which the area or the diameter of the pupil increases with time corresponds to mydriasis, while an interval in which the area or the diameter of the pupil decreases with time corresponds to myosis. That is, the interval in which the pupil size increases with time corresponding to mydriasis, while the interval in which the pupil size decreases with time corresponds to myosis.

Note that the change amount of the pupil size due to light reflex is generally about several times as large as the change amount of the pupil size due to emotion and becomes a great factor for the entire change amount of the pupil size. In order to reduce changes due to light reflex and convergency reflex and make attention easily paid to only a component related to the perception of a salient sound, the brightness of a screen presented to the subject and a distance from the screen to the subject when a pupil radius is acquired are made constant.

[Iris Information Acquisition Unit 230]

In S230, the iris information acquisition unit 230 acquires and outputs the time series of iris information (hereinafter called the time series of first iris information and the time series of second iris information) that correspond to the first time interval and the second time interval, respectively, and express the iris size of the subject. The iris size may be acquired by the same method as that of the pupil size in S220. Accordingly, any value such as the z-score of an iris radius, the value itself of the iris radius, the area of the iris, and the diameter of the iris may be used as the iris size so long as the value corresponds to the iris size.

[Pupil Feature Value Calculation Unit 240]

In S240, the pupil feature value calculation unit 240 calculates, using the time series of the first pupil information and the time series of the second pupil information acquired in S220 and the time series of the first iris information and the time series of the second iris information acquired in S230 as inputs, the ratio of the pupil information to the iris information (the pupil information/the iris information) as a pupil feature value from the pupil information and the iris information included in the time series of the first pupil information and the time series of the first iris information, respectively, and the pupil information and the iris information included in the time series of the second pupil information and the time series of the second iris information, respectively. The pupil feature value calculation unit 240 generates and outputs the time series of pupil feature values (hereinafter called the time series of a first pupil feature value and the time series of a second pupil feature value) corresponding to the first time interval and the second time interval, respectively. Note that the pupil information and the iris information are preferably acquired by the same method as with the pupil feature value calculation unit 140.

[Pupil Change Feature Value Extraction Unit 250]

In S250, the pupil change feature value extraction unit 250 extracts, using the time series of the first pupil feature value and the time series of the second pupil feature value generated in S240 as inputs, feature values (hereinafter called a first pupil change feature value and a second pupil change feature value) that correspond to the first time interval and the second time interval, respectively, and express a change in the pupil size of the subject from the time series of the first pupil feature value and the time series of the second pupil feature value, and outputs the extracted feature values.

The feature values (pupil change feature values) expressing a change in the pupil size can also be indexes for estimating saliency. In other words, the feature values are feature values expressing a change in the pupil size in an interval in which mydriasis occurs among the time series of the pupil feature values (the time series of the feature values expressing the pupil size). Specifically, the feature values are feature values including at least any one or more of an average speed V of mydriasis, an amplitude A of the mydriasis, and a damping coefficient ζ obtained by modeling the time series of a pupil radius where the mydriasis occurs as the step response of a position control system. The amplitude A is a difference in pupil radius between a local maximum point and a local minimum point (see FIG. 7). The average speed V of the mydriasis is obtained by dividing the amplitude A by a rising time Tₚ. The rising time Tₚ is a time from the local maximum point to the local minimum point (see FIG. 7). For example, the pupil change feature value extraction unit 250 detects a local maximum point and a local minimum point from the time series of the pupil feature values and calculates the amplitude A, the average speed V, and the rising time Tₚ using the detected local maximum point and the local minimum point. At this time, the pupil change feature value extraction unit 250 may be configured to calculate only an amplitude of a constant value or more.

Note that the myosis and the mydriasis show the features of a servo system and a step-wise saccade can be described as the step response of an area control system (tertiary delay system) In the present embodiment, it is considered that the step-wise saccade is approximated as the step response of a position control system (secondary delay system). Using a natural angular frequency as ωₙ, the step response of the position control system is expressed by the following formula.

$$G ( s ) = \frac{A {ω}_{n}^{2}}{{s}^{2} + 2 {ζω}_{n} s + {ω}_{n}^{2}} \\ y ( t ) = A \{ 1 - {e}^{- {ζω}_{n} t} ( \frac{ζ}{\sqrt{1 - {ζ}^{2}}} sin {ω}_{d} t + cos {ω}_{d} t ) \} \\ {y}^{′} ( t ) = \frac{A {ω}_{n}}{\sqrt{1 - {ζ}^{2}}} {e}^{- ζ {ω}_{n} t} sin {ω}_{d} t \\ {ω}_{d} = {ω}_{n} \sqrt{1 - {ζ}^{2}} \qquad [ Formula 1 ]$$

Here, G(s) expresses a transfer coefficient, y(t) expresses a position, and y′ (t) expresses a speed. On the basis of the following formula, the ratio of a time Tₐ at which the speed becomes maximum to a rising time Tₚ is used (see FIG. 10) to derive the damping coefficient ζ.

$$tan ( \frac{{T}_{a}}{{T}_{p}} π ) = \frac{\sqrt{1 - {ζ}^{2}}}{ζ} \qquad [ Formula 2 ]$$

Then, each of the damping coefficient ζ and the natural angular frequency ωₙ is expressed by the following formula.

$$ζ = \frac{1}{\sqrt{1 + {tan}^{2} ( \frac{{T}_{a}}{{T}_{p}} π )}} \\ {ω}_{n} = \frac{π}{{T}_{p} \sqrt{1 - {ζ}^{2}}} \qquad [ Formula 3 ]$$

Here, t is an index expressing a time, and s is a parameter (complex number) based on a Laplace transform. The natural angular frequency ωₙ corresponds to an index expressing a response speed in a change in the pupil size, and the damping coefficient ζ corresponds to an index corresponding to the vibratility of a response in a change in the pupil size.

Note that when the mydriasis is included in the first time interval for a plurality of times, the representative value of an average speed V, an amplitude A, or a damping coefficient ζ calculated for each mydriasis is used as the feature of the mydriasis corresponding to the first time interval. The representative value is, for example, an average value, a maximum value, a minimum value, a value corresponding to the first mydriasis, or the like. Particularly, it is preferable to use the average value. Further, when the mydriasis is not included in the first time interval even once, the representative value of an average speed V, an amplitude A, or a damping coefficient ζ calculated for the mydriasis just after the first time interval (the mydriasis occurring after the first time interval in terms of time and occurring at a time closest to the first time interval) is used as the feature of the mydriasis corresponding to the first time interval. That is, information on the pupil size corresponding to the first time interval is acquired so as to include the mydriasis at least once. The same applies to the second time interval.

[Saliency Estimate Unit 260]

In S260, the saliency estimate unit 260 estimates the degree of saliency of a prescribed sound (target sound) on the basis of the degree of the difference between the first pupil change feature value and the second pupil change feature value extracted in S250.

Specifically, when the feature values are the average speed V of the mydriasis and the amplitude A of the mydriasis, it is estimated that the saliency is higher as the first pupil change feature value is larger than the second pupil change feature value and the difference between the first pupil change feature value and the second pupil change feature value is larger.

Alternatively, when the feature value is the damping coefficient ζ of the mydriasis, it is estimated that the saliency is higher as the first pupil change feature value is smaller than the second pupil change feature value and the difference between the first pupil change feature value and the second pupil change feature value is larger.

The above estimation results are based on the fact that the establishment of the following corresponding relationships between the damping coefficient ζ of the mydriasis, the average speed V of the mydriasis, and the amplitude A of the mydriasis and the saliency of a target sound become clear from an experiment.

(1) The saliency is larger as the average speed V of the mydriasis increases.
(2) The saliency is larger as the amplitude A of the mydriasis increases.
(3) The saliency is larger as the damping coefficient ζ of the mydriasis decreases.

Note that any one of the average speed V, the amplitude A, and the damping coefficient ζ may be singly used or the feature values may be used in combination. For example, any two of the feature values are only required to be satisfied, or all the three feature values are only required to be satisfied. That is, the degree of saliency of a target sound may be estimated on the basis of the degree of a difference in each of one or more of the feature values of the average speed V, the amplitude A, and the damping coefficient ζ for the first time interval and the second time interval.

Since the average speed V and the amplitude A of the mydriasis reflect the active strength of a sympathetic nerve, it is presumed that the average speed V and the amplitude A are correlated with the saliency of a sound. The damping coefficient (is an index corresponding to the vibratility of a response when the mydriasis is regarded as the step response of a position control system (secondary delay system). When a sound (salient sound) having high saliency is listened to, the awareness of the sound is raised. As a result, it is presumed that a temporal influence is exerted on the nerve center of the brain or a musculus dilator pupillae (or a musculus sphincter pupillae) related to the control of the pupil and can be observed as a change in vibratility (damping coefficient) of a response.

According to the findings, that is, the corresponding relationships of (1) to (3), the saliency estimate unit 260 estimates the saliency of a prescribed sound on the basis of the degree of the difference between the first pupil change feature value that is the feature value of a change in the pupil size in the first time interval in which the prescribed sound is presented so as to be audible and the second pupil change feature value that is the feature value of a change in the pupil size in the second time interval in which the prescribed sound is not audible.

Specifically, when the feature value is the damping coefficient ζ of the mydriasis, it is estimated that the saliency of a sound is high when the first pupil change feature value is smaller than the second pupil change feature value. Further, it is estimated that the degree of saliency of a sound is higher as the absolute value of the difference between the first pupil change feature value and the second pupil change feature value is larger. If a sound different from a prescribed sound (the sound of the first time interval) is presented in the second time interval, it is estimated that the saliency of a sound presented in a time interval corresponding to a smaller one of the first pupil change feature value and the second pupil change feature value has higher saliency.

When the feature value is the average speed V of the mydriasis or the amplitude A of the mydriasis, it is estimated that the saliency of a sound is high when the first pupil change feature value is larger than the second pupil change feature value. Further, it is estimated that the degree of saliency of a sound is higher as the absolute value of the difference between the first pupil change feature value and the second pupil change feature value is larger. If a sound different from a prescribed sound (the sound of the first time interval) is presented in the second time interval, it is estimated that the saliency of a sound presented in a time interval corresponding to a larger one of the first pupil change feature value and the second pupil change feature value has higher saliency.

According to the embodiment of the present invention, it is possible to estimate the degree of saliency of a prescribed sound for a subject on the basis of a change in pupil size. At this time, it is possible to correctly estimate the change in the pupil size without being susceptible to the positional relationship between a camera and an eyeball by the use of a pupil feature value that is the ratio of pupil information to iris information.

### APPENDIX

As, for example, a single hardware entity, the device of the present invention has an input unit to which a keyboard or the like is connectable, an output unit to which a liquid crystal display or the like is connectable, a communication unit to which a communication device (for example, a communication cable) that is capable of communicating with the outside of the hardware entity is connectable, a CPU (Central Processing Unit, which may include a cache memory, a resistor, or the like), a RAM or a ROM that is a memory, an external storage device that is a hard disk, and a bus that connects the input unit, the output unit, the communication unit, the CPU, the RAM, the ROM, and the external storage device to each other so as to allow data exchange therebetween. Further, a device (drive) or the like that can perform the reading/writing of information on a recording medium such as a CD-ROM may be provided in the hardware entity where necessary. As a physical body including such hardware resources, a general-purpose computer or the like is available.

In the external storage device of the hardware entity, programs necessary for realizing the functions described above and data or the like necessary for processing the programs are stored (the programs may be stored in, for example, the ROM that is a read-only storage device rather than being stored in the external storage device). Further, data or the like obtained by the processing of the programs is appropriately stored in the RAM, the external storage device, or the like.

In the hardware entity, respective programs stored in the external storage device (or the ROM or the like) and data necessary for the processing of the respective programs are read in a memory where necessary and appropriately interpreted and processed by the CPU. As a result, the CPU realizes the prescribed functions (the respective constituting elements expressed as *** unit, *** means, or the like in the above description).

The present invention is not limited to the embodiments described above but may be appropriately modified without departing from the spirit of the present invention. Further, the processing described in the above embodiments may be performed not only chronologically along the described orders but also parallelly or separately according to the processing performance of the device that performs the processing or where necessary.

As described above, when the processing functions of the hardware entity (the device of the present invention) described in the above embodiments are realized by a computer, the processing contents of the functions that are to be provided in the hardware entity are described by a program. Then, when the program is performed by the computer, the processing functions of the above hardware entity are realized on the computer.

The program in which the processing contents are described can be recorded on a computer-readable recording medium. As a computer-readable recording medium, any type of a recording medium such as a magnetic recording device, an optical disc, a magneto-optical recording medium, and a semiconductor memory can be used. Specifically, for example, as a magnetic recording device, a hard disk device, a flexible disk, a magnetic tape, or the like can be used. Further, as an optical disc, a DVD (Digital Versatile Disc), a DVD-RAM (Random Access Memory), a CD-ROM (Compact Disc Read Only Memory), a CD-R (Recordable)/RW (ReWritable), or the like can be used. Further, as a magneto-optical recording medium, a MO (Magneto-Optical disc) or the like can be used. Further, as a semiconductor memory, an EEP-ROM (Electronically Erasable and Programmable-Read Only Memory) or the like can be used.

Further, the circulation of the program is performed by, for example, selling, transferring, leasing, or the like of a transportable recording medium such as a DVD and a CD-ROM on which the program is recorded. In addition, the circulation of the program may be performed in such a manner that the program is stored in the storage device of a server computer in advance and the program is transferred from the server computer to other computers via a network.

A computer that performs such a program first temporarily stores, for example, a program recorded on a transportable recording medium or a program transferred from a server computer in its own storage device. Then, when performing the processing, the computer reads the program stored in the own storage device and performs processing according to the read program. Further, as another mode to perform a program, the computer may directly read a program from a transportable recording medium and perform processing according to the program. In addition, every time a program is transferred from a server computer to the computer, the computer may successively perform processing according to the received program. Further, the computer may be configured to perform the above processing by a so-called ASP (Application Service Provider) type service in which a program is not transferred to the computer and processing functions are realized only by executing instructions and result acquisition. Note that a program in the present mode includes information that is subjected to the processing of an electronic calculator and corresponds to the program (such as data that is not a direct instruction to a computer but has the property of stipulating the processing of the computer).

Further, a prescribed program is performed on a computer to constitute a hardware entity in the mode, but at least a part of the processing contents may be realized in terms of hardware.

1. A pupil feature value extraction apparatus comprising: a pupil information acquisition unit that acquires pupil information expressing a pupil size of a subject from an image of an eyeball of the subject; an iris information acquisition unit that acquires iris information expressing an iris size of the subject from the image; and a pupil feature value calculation unit that calculates a ratio of the pupil information to the iris information as a pupil feature value.

2. The pupil feature value extraction apparatus according to claim 1, wherein the pupil information acquisition unit acquires the pupil information using two points on an edge of a pupil region in the image, and the iris information acquisition unit acquires the iris information using two points on an edge of an iris region in the image.

3. The pupil feature value extraction apparatus according to claim 2, wherein the pupil information acquisition unit or the iris information acquisition unit extracts a region having intensity smaller than or equal to a prescribed threshold as the pupil region or the iris region in a binary image obtained by converting the image, calculates a gray value of pixels on a line passing through a center of the pupil region or the iris region, extracts a peak of a first derivative of the gray value, and extracts a zero cross point of a second derivative of the gray value near the peak as a point on the edge of the pupil region or the iris region.

4. A pupil feature value extraction method comprising: a pupil information acquisition step of acquiring, by a pupil feature value extraction apparatus, pupil information expressing a pupil size of a subject from an image of an eyeball of the subject; an iris information acquisition step of acquiring, by the pupil feature value extraction apparatus, iris information expressing an iris size of the subject from the image; and a pupil feature value calculation step of calculating, by the pupil feature value extraction apparatus, a ratio of the pupil information to the iris information as a pupil feature value.

5. A non-transitory computer-readable storage medium which stores a program for causing a computer to function as the pupil feature value extraction apparatus according to claim 1.
